(19) United States
(12) Patent Application Publication
(10) Pub. No.: US 2024/0296933 A1
(43) Pub. Date: Sep. 5, 2024

(54) SYSTEMS AND METHODS OF REMOTE THERAPEUTIC MONITORING AND OPTIMIZATION OF HEALTHCARE INTERVENTIONS

(71) Applicant: Steven Merahn, Orchard Lake, MI (US)

(72) Inventor: Steven Merahn, Orchard Lake, MI (US)

(21) Appl. No.: 18/177,281

(22) Filed: Mar. 2, 2023

(51) Int. Cl. G16H 20/70; A61B 5/16; G16H 50/70

(52) U.S. Cl. CPC G16H 20/70; A61B 5/16; G16H 50/70

(57) ABSTRACT

The present invention relates to methods and systems to precisely define and track benchmarks, progress, and endpoints of treatment or interventions associated with behavioral health conditions and health-related behaviors in order to optimize outcomes. The present invention is specifically designed to improve the rate of improvement of the behavioral health of patients.

### BACKGROUND OF THE INVENTION

The present invention relates to healthcare and, more particularly, to methods and systems to precisely define and track benchmarks, progress and endpoints of treatment or interventions associated with behavioral health conditions and health-related behaviors to optimize outcomes.

At the present, healthcare does not have a standardized process or method for describing or documenting behavior-based outcomes associated with medical or behavioral treatments or interventions, including but not limited to medication dosing, frequency, or type. Tragically, this has impeded the capacity of these systems of care to understand the value or impact of such therapies and optimize associated treatment strategies. There are no devices or systems that allow for continuous, consistent tracking of behavioral targets or outcomes in such a way as to support therapeutic optimization.

As can be seen, there is a need for a system and method that is specifically designed to improve the rate of improvement of patients' behavioral health. The present invention provides (1) a specific behavioral rubric that uses behavior as the basis of an assessment and optimization schema, (2) a remote therapeutic monitoring platform, (3) the use of semi-logarithmic analytics to standardize the analytics.

### SUMMARY OF THE INVENTION

In one aspect of the present invention, a method of treating a behavioral issue of a patient, the method includes following the steps: defining, by way of at least one processor, an initial graphical point associated with the behavioral issue of the patient; administering a treatment to the patient, wherein the treatment comprises a type, a dosage, and a frequency of a medicament capable of treating the behavioral issue; receiving, by way of the at least one processor, one or more time-dependent graphical points, wherein each one or more time-dependent graphical points defines an observable human behavior of the patient in terms of an action data and a contextual data; and graphically analyzing, by way of the at least one processor, a rate of change defined by a connection between the initial graphical point and the one or more time-dependent graphical points.

In another aspect of the present invention, the method of treating a behavioral issue of a patient further includes wherein the context data comprises at least three of the following: a time, a physical location, a target individual, and an object associated with the observed human behavior, wherein the action data comprises at least one action associated with the behavioral issue, wherein said graphical analysis is logarithmic; further including graphing, by way of the at least one processor, a semi-log chart of the graphical analysis; displaying, by way of the at least one processor, the semi-log chart on a user interface operatively associated with the at least one processor; a rules engine operatively associated with the at least one processor, wherein the rules engine is configured to generate a support alert after determining that a threshold rate of change is exceeded by the rate of change defined by the graphical analysis, wherein the threshold rate of change is based, in part, on evidence-based guidelines retrievable by the rules engine; and a support platform operatively associated with the at least one processor, wherein the support platform receives the support alert.

Behavior can be described in two ways: its function or operation, which allows clinicians to understand the behavior in respect to the environment, and its form—also known as its topography—which allows clinicians to understand what the behavior looks like. The same topography can have different functions in different situations and individuals.

Topography and function represent two very different ways of describing a behavior. For example, if a child throws a tantrum, to explain the topography of the behavior, it would not be enough for a teacher to simply say “the child threw a tantrum.” A topographical definition might state: “The child threw herself on the floor, and kicked and screamed in a high-pitched voice. The child did not make physical contact with other individuals, furniture, or other items in the environment.” The functional or operational description, by contrast, would be open to interpretation: “Lisa became angry, swung her arms and tried to strike other children and the teacher while screaming in that high-pitched voice she often uses.” Each description could be defined as a “tantrum,”

Understanding function or operative definition can be valuable because it refers to the reasons and contingencies associated with actions or behaviors which can be leveraged to therapeutic advantage. However, topography-based definitions are useful when the clinician does not have direct, reliable, or easy access to the underlying reason for target behavior, because it allows for consistent measurement of response to tests of various therapeutic interventions.

Functional or operational definitions of behaviors or goals are highly variable and usually do not meet reliable standards for measurement nor have ‘between-group’ translational validity. Consequently, the present invention was developed that includes a taxonomy for the development of topographic definitions of behaviors, health-related goals, or symptomatology, and a method by which those definitions can be used to define a baseline against which the effect of therapeutic interventions can be assessed using a standardized semi-logarithmic ratio chart to allow for comparative analysis of progress towards goal. The topographic taxonomy of the present invention specifies the exact target an observer would detect and count without further definition, providing better detection accuracy and, when used for the continuous, real-time observation periods and analyzed using the semi-logarithmic ratio chart, can detect meaningful changes associated with a therapeutic intervention, support rapid, accurate decisions by clinicians as to whether a change is needed.

Accordingly, the present disclosure provides a system or method that provides clinicians and patients (and/or their families) with: (1) a topographic taxonomy to precisely describe and define observable and measurable pinpoint targets for intervention; (2) a structured method of measuring the frequency, duration, latency (time between behaviors), or context of the target at baseline, and subsequent to the addition of a medical or behavior therapeutic intervention; (3) a method of recording, tracking and analyzing data with semi-logarithmic algorithm and analyze the rate of change; and (4) the capacity to add logic analytics to generate insights into the rate of change and progress towards goals and recommending clinical decisions associated with optimizations of dose, timing, and type of medications.

As will be readily apparent to those with skill in the art, the present invention has many potential use cases. For example, it may be used for, such as, but not limited to: medication treatment, such as for Tardive Dyskensia, Obsessive Compulsive Disorder, and Oppositional Deficient Disorder; autism, such as for Severe Problem Behavior and skills acquisition; ADHD, such as for inattention/attention span, hyperactivity/motor restlessness, impulsivity, and distractibility; anxiety, such as for panic, phobias, and general anxiety; compulsive behaviors, such as for obsessive thoughts, rituals/routines, stereotypies (repetitive movements or utterances), and perseveration; unstable mood, such as for agitation, irritability, aggression/hostility, lability/emotional control, tantrums, and explosive behavior/rage reactions; self-injurious behavior; depressed mood, such as for loss of interest, unhappiness/miserableness, under-activity, and suicidality; sleep issues, such as for insomnia, hypersomnia, early morning awakening, daytime drowsiness; eating patterns, such as for appetite control, anorexia, and bulimia; elevated mood, such as for grandiosity, intrusiveness, hypersexuality, and bi-polar cycling; psychotic issues, such as for hallucinations, delusions, and self-talk; movements, such as for motor restlessness, catatonia (‘stuck’), and tics; neuropsychological (e.g., dementia), such as for concentration, memory, wandering, and safety; alcohol misuse; drug misuse; sexual issues/problems; and psychosomatic complaints.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a block diagram 10 of a method and system of the present invention.

FIG. 2 is a flow chart 20 of a therapeutic treatment method of the present invention.

FIG. 3 is a diagrammatical view of chart of an exemplary embodiment of the present invention, whereon logarithmic and semi-logarithmic values can be plotted and frequency analysis can be calculated from.

FIG. 4 is a diagrammatical view of output of an exemplary embodiment of the present invention.

### DETAILED DESCRIPTION OF THE INVENTION

With this overview in mind, and turning now to a more detailed discussion in conjunction with the attached figures, certain techniques of the present disclosure is independent of medium and may be implemented in a variety of ways including but not limited to paper, digital spreadsheets, or computing devices such as a PC, laptop, tablet, smartphone or other device capable of executing computer-executed instructions stored on a non-transient medium, e.g., memory, such as RAM, ROM, EPROM, flash memory and so on. Thus, the execution of steps in a process flow is by way of computer-execution of such steps, e.g., via a processor configured to retrieve the corresponding instructions from memory and execute them.

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. It may be evident, however, that the present disclosure may be practiced without some of these specific details.

Broadly, one embodiment of the present invention is a method and system to precisely define and track behavioral and/or health goals to optimize outcomes for the patient.

Certain aspects of the system of the present invention may include at least one computer with a user interface. The computer may include at least one processing unit coupled to a form of memory. The computer may include, but is not limited to, a microprocessor, a server, a desktop, laptop, and smart device, such as, a tablet and smart phone. The computer includes a program product including a machine-readable program code for causing, when executed, the computer to perform steps. The program product may include systemic software which may either be loaded onto the computer or accessed by the computer. The loaded systemic software may include an application on a smart device. The systemic software may be accessed by the computer using a web browser. The computer may access the systemic software via the web browser using the internet, extranet, intranet, host server, internet cloud and the like.

As stated above, healthcare does not have a standardized process or method for describing or documenting behavior-based outcomes associated with medical or behavioral treatments or interventions, such as changes in medication dosing, frequency, or type. This has impeded the capacity of current systems of care to understand the value or impact of such therapies and optimize associated treatment strategies. The present invention claimed here solves this problem. The system and methods, in accordance with the present invention, provide a framework for defining and describing targets or aspirations for behavioral outcomes, counting the frequency, duration, latency, or context of behaviors at baseline, tracking changes in behaviors associated with therapies or interventions, and calculating the rate of change or progress and providing feedback as to when therapeutic interventions should be changed in order to optimize the outcomes for the patient, such as changing medication dosing frequency or type.

The method described herein allows for more accurate standardized information architecture to define therapeutic endpoints and a method of continuous, consistent tracking of targets or outcomes in such a way as to support therapeutic optimization. No other existing systems or methods improve inter-rater consistency or reliability of behavioral targets, nor methods to accurately assess the rate of change in such a way as to support clinical decision-making, including but not limited to changing medication dose, frequency of dosing, or type of medication.

The present invention provides a novel standardized framework for defining the goal and/or outcome associated with an intervention, a method of tracking progress towards one or more goals based on that definition, and a standardized approach to tracking rate of change. As a result, previous issues, such as inconsistency in targeting, variability in the data model for analytics even when data is collected, lack of capacity to link the analysis to clinical decision support, can effectively addressed. The present invention is specifically designed to improve the rate of improvement of patients' behavioral health, based on the capacity to generate more sensitive and specific data on the different between expected and actual rate of change as defined by established guidelines and evidence (e.g., Food and Drug Administration Drug Labels), electronic medical record data or claims, real-world evidence (disruptions in school performance), patient-defined determinations, and others.

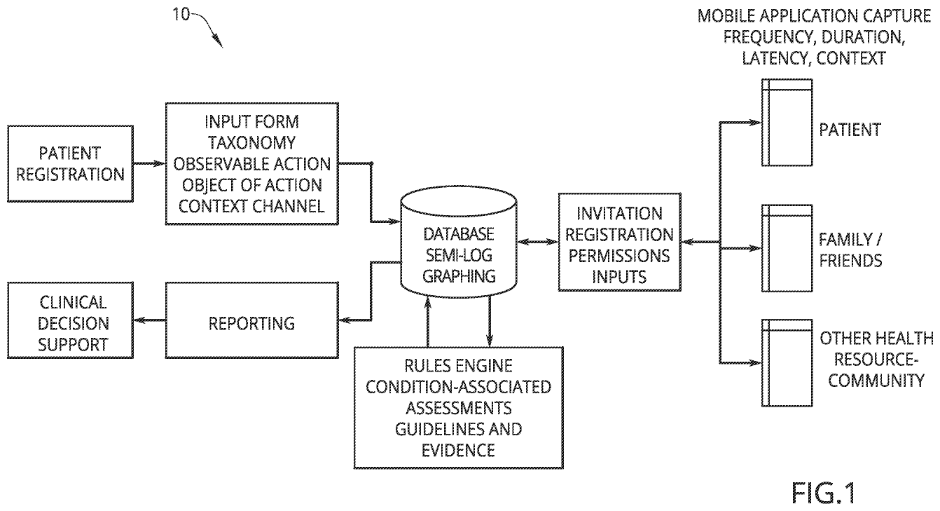

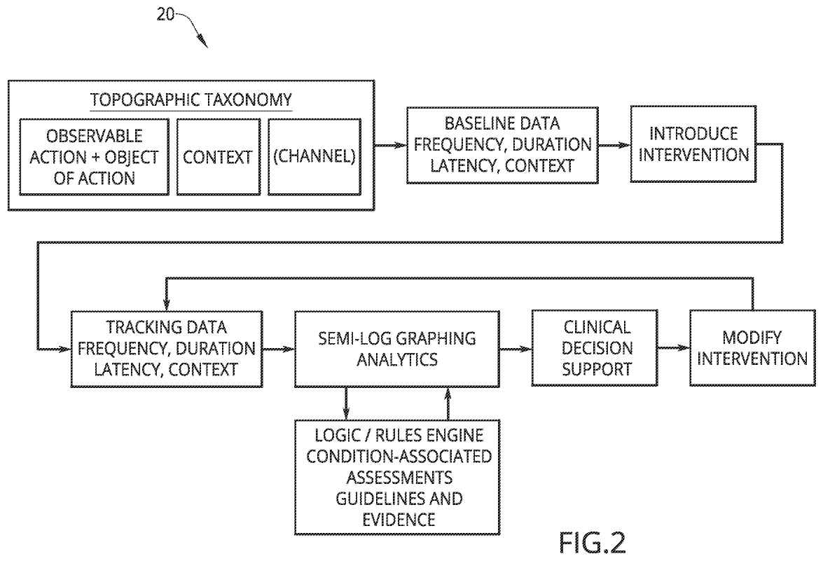

Referring now to FIGS. 1 and 2, a system of the present invention includes a website, portal, mobile application, or an area (generally referred herein to as a clinician platform) functions within an electronic medical record that provides a framework for defining a patient-specific behavioral target. This clinician platform functions as an input platform where the initial behavior of a patient is defined where therapeutic intervention (e.g., medication) is added and documented. This data feeds a user mobile application/platform that shares the behavioral target and associated standardized assessments with the patient and their personal resource community so that data can be gathered on the baseline frequency, duration, latency, or context of the behavior and condition of the patient. Once the therapeutic intervention is started, the patient and their resource community are notified as to that start date of the intervention period and they can continue to document the targeted behavior. The data from the user platform is populated in a data warehouse for storage. This data, once collected, can be analyzed using a semi-logarithmic model. Specifically, the slope of the curve can be analyzed and shared with the prescribing provider to determine the effectiveness of the current therapeutic intervention.

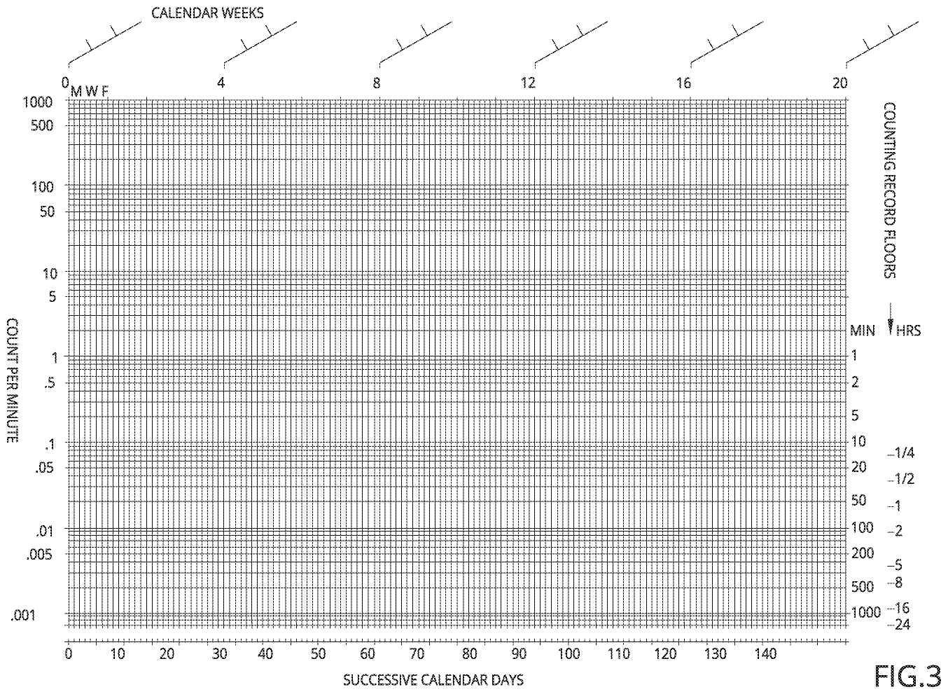

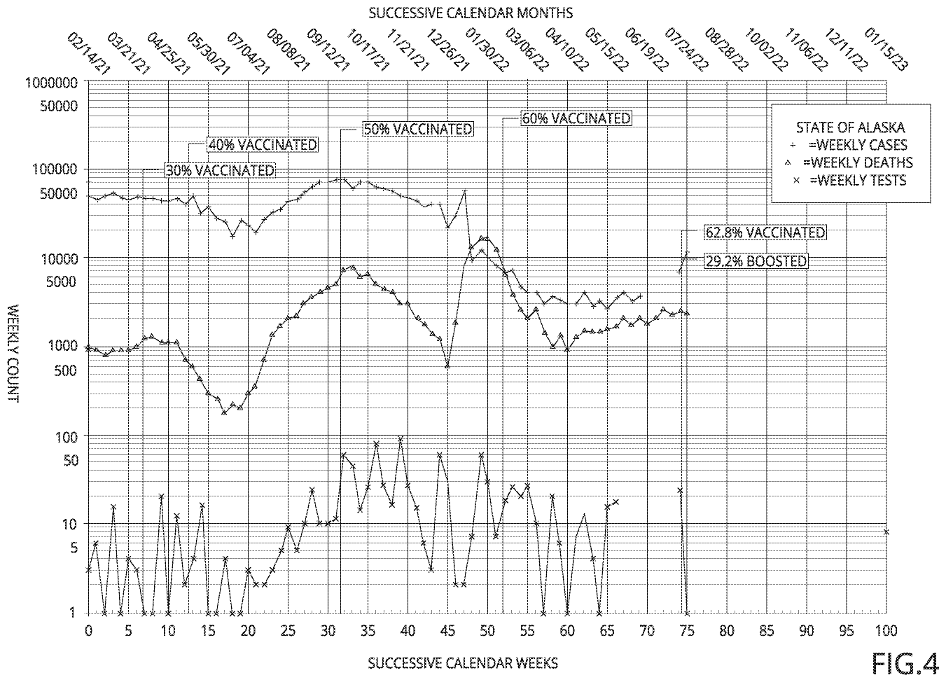

Referring to FIGS. 3 and 4, the combination of the topographic taxonomy as the basis to define and describe the target objective for change (behavior, symptom) the semi-log frequency analysis shows the direction and speed of behavior change. The steeper the curve, the greater the effect of the intervention. Without detailed taxonomy and the semi-log analytics there is no way of knowing, or communicating to others, the precise speed of change of any intervention. The semi-log analytics also support a way of standardizing reporting and comparative effectiveness of interventions to patients, families, researchers, insurance companies, and others who have a vested interest in rate of change associated with interventions or cohorts of individuals with diverse demographic or other characteristics.

Decision support is generated to the provider (in accordance with decision support rules that drive the outputted decision support) if progress towards the targeted outcome is adequate, with recommended changes in treatment strategies. By way of example, a medication is known to start reducing symptoms within 72 hours of first dose, but the slope of the curve for the targeted symptom is flat or less than 25% improvement that that time, it would inform the prescribing physicians decision to increase the dose or switch medications. Similarly, if the patients slope dropped after a period of time of demonstrated effectiveness, it would trigger an inquiry as to whether there is a new issues with medication regimen adherence

FIG. 1 generalizes this process, with the left side of the block diagram illustrating the “clinician platform” described above, the right side of the block diagram illustrating the “user platform” side described above, and the center of the block diagram illustrating the “data warehouse” described above.

All of the elements of the present invention are fully integrated to form a system for improving the rate of improvement of patients' behavioral health. The definitions of behavioral targets are specifically structured to be observable in frequency, duration, latency, or context. Saying that a patient “is aggressive” isn't measurable; but a pinpoint, such as “patient hits parent when TV is turned off” is measurable. The topographic taxonomy invented and described herein combines an action-verb and an object of that action (“hits parent”) and added context (when, where, with whom, with what). Sometimes the sensory channel is included (when seen, when heard).

### Initial Behavioral Points

Medication Treatment-related, examples
   Behavioral
      Tardive Dyskensia
      Obsessive Compulsive Disorder
      Oppositional Defiant Disorder
   Medical, Symptom reduction
      Congestive Heart Failure
      Chronic Obstructive Pulmonary Disease
      Migraine
Autism-related, examples
   Severe Problem Behavior
   Skills acquisition
ADHD-related, examples
   Inattention/attention span
   Hyperactivity/Motor restlessness
   Impulsivity
   Distractibility
Anxiety-related
   Anxiety
   Panic
   Phobias
Compulsive behaviors
   Obsessive thoughts
   Rituals/routines
   Stereotypies (repetitive movements or utterances)
   Perseveration
Unstable Mood
   Agitation
   Irritability
   Aggression/Hostility
   Lability/Emotional Control
   Tantrums
   Explosive behavior/Rage reactions
Self-injurious behavior
Depressed mood
   Loss of interest
   Unhappy/miserable
   Under-activity
   Suicidality
Sleep issues
   Insomnia
   Hypersomnia
   Early morning awakening
   Daytime drowsiness
Eating patterns
   Appetite control
   Anorexia
   Bulimia
Elevated mood
   Grandiosity
   Intrusiveness
   Hypersexuality
   Bi-polar cycling
Psychotic-related
   Hallucinations
   Delusions
   Self-talk
Movement-related
   Motor restlessness
   Catatonia (‘stuck’)
   Tics
Neuropsychological (e.g., dementia)
   Concentration
   Memory
   Wandering
   Safety
Alcohol misuse
Drug abuse
Sexual issues/problems
Psychosomatic complaints

Further, the choice of semi-logarithmic analytics is based on its sensitivity to provide insights into frequency changes and accuracy in recommending clinical decisions associated with optimizations of dose, timing, and type of medications. The logic associated with the present invention is derived from evidence-based guidelines and evidence as to first, second and third-line therapeutic decision-making.

An exemplary method of building out the system of the present invention may include the following. First, the input screens for defining the behavioral targets are built. The database is structured to store the behavioral targets and the details on planned interventions. A mobile application or other platform is developed to share the framework for defining behavioral targets, and associated standardized assessments, with members of the patient personal resource-community, along with the input screen and permissions associated with being a member of an individual's resource community. Registration and service level agreements are created for members of the resource community, as well as the process by which their devices are connected to the database (e.g., via permissions over a network).

In the database, the semi-log analytics may be coded as algorithms and run in real-time as data is entered. The results of the analytics may be analyzed as changes in the slope of the curve. Based on a rules engine that combines expected results with known evidence-based guidelines and evidence, the specific slope generates a decision support alert for the physician to make a change in treatment. As those with skill in the art will appreciate, the same system and methods could be applied to any observable or measurable non-behavioral patient outcomes such as treatment of congestive heart failure, chronic obstructive pulmonary disease, substance abuse, or dental hygiene, and the like (as discussed above).

A physician or other healthcare professional would deploy this system in conjunction with their therapeutic recommendations. Using a shared decision-making model, they would work with the patient and/or their family to define the goals/endpoints/outcomes and deploy the remote monitoring component to track progress. The data is collected and analyzed and presented to the physician for decision-making.

As described in detail above, the present invention is, in part, a sequence of rigorous instructions for remote therapeutic monitoring and optimization of healthcare interventions based on real-world evidence. In accordance with the present invention, a novel topographic taxonomy to precisely describe and define observable and measurable human behavior (broadly defined: including but not limited to actions, skills, strengths, reactions, responses, mannerisms, physical, motor, sensory or psychological) is disclosed. Further disclosed is a structured method of measuring the frequency, duration, latency, or context of the target behavior in the real-world. Further disclosed is a method of recording this data at baseline and after any one of number of therapeutic interventions (e.g., medications, therapies) designed to change the behavior. Further disclosed is a standardized method of analyzing the data with using semi-logarithmic scales to analyze the rate of change. Further disclosed is a method that has the capacity to add logical insights associated with the rate of change and further track the effect of changes in dose, timing, and type of interventions on progress towards goal. As those with skill in the art will appreciate, while the present invention is preferably implemented using technology, it is not necessarily dependent thereon. For example, while a recordation method could be done a mobile application loaded on a mobile device, it could be done via a form. Further, a method of analyzing data could be done by a computer or it could also be done by slide rule.

While one or more preferred embodiments are disclosed, many other implementations will occur to one of ordinary skill in the art and are all within the scope of the invention. Each of the various embodiments described above may be combined with other described embodiments to provide multiple features. Furthermore, while the foregoing describes a few separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

The computer-based data processing system and method described above is for purposes of example only and may be implemented in any type of computer system or programming or processing environment, or in a computer program, alone or in conjunction with hardware. The present invention may also be implemented in software stored on a computer-readable medium and executed as a computer program on a general-purpose or special-purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system or run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

What is claimed is:

1. A method of treating a behavioral issue of a patient, the method comprising the steps of:
defining, by way of at least one processor, an initial graphical point associated with the behavioral issue of the patient;
administering a treatment to the patient, wherein the treatment comprises a type, a dosage, and a frequency of a medicament capable of treating the behavioral issue;
receiving, by way of the at least one processor, one or more time-dependent graphical points, wherein each one or more time-dependent graphical points defines an observable human behavior of the patient in terms of an action data and a contextual data; and
graphically analyzing, by way of the at least one processor, a rate of change defined by a connection between the initial graphical point and the one or more time-dependent graphical points.

2. The method of claim 1, wherein the context data comprises at least three of the following: a time, a physical location, a target individual, and an object associated with the observed human behavior.

3. The method of claim 2, wherein the action data comprises at least one action associated with the behavioral issue.

4. The method of claim 3, wherein said graphical analysis is logarithmic.

5. The method of claim 4, further comprising graphing, by way of the at least one processor, a semi-logarithmic chart of the graphical analysis.

6. The method of claim 4, further comprising displaying, by way of the at least one processor, the semi-logarithmic chart on a user interface operatively associated with the at least one processor.

7. The method of claim 1, further comprising a rules engine operatively associated with the at least one processor, wherein the rules engine is configured to generate a support alert after determining that a threshold rate of change is exceeded by the rate of change defined by the graphical analysis, wherein the threshold rate of change is based, in part, on evidence-based guidelines retrievable by the rules engine.

8. The method of claim 1, further comprising a support platform operatively associated with the at least one processor, wherein the support platform receives the support alert.
